(19) United States
(12) Patent Application Publication
(10) Pub. No.: US 2023/0346712 A1
(43) Pub. Date: Nov. 2, 2023

(54) MEDICINE LOADED NANO-CAPSULE ADDITIVE PMMA CEMENT

(71) Applicant: ATATÜRK ÜNIVERSITESI BILIMSEL ARASTIRMA PROJELERI BIRIMI, Yakutiye, Erzurum (TR)

(72) Inventors: Kutsi TUNCER, Yakutiye, Erzurum (TR); Ömer GÜNDOGDU, Yakutiye, Erzurum (TR); Abdullah BASÇI, Yakutiye, Erzurum (TR); Bahri GÜR, Igdir (TR); Onur SENOL, Yakutiye, Erzurum (TR); Muhammet Raci AYDIN, Igdir (TR)

(21) Appl. No.: 17/905,867

(22) PCT Filed: Aug. 27, 2021
(86) PCT No.: PCT/TR2021/050871

§ 371 Date: Sep. 8, 2022

(30) Foreign Application Priority Data
Aug. 31, 2020 (TR) 2020/13709

(51) Int. Cl. A61K 9/51; A61K 38/14; A61K 31/7036; A61K 31/5377; A61L 24/00

(52) U.S. Cl. CPC A61K 9/5138; A61K 38/14; A61K 9/5192; A61K 31/7036; A61K 31/5377; A61L 24/0015; A61L 24/0094; A61L 2400/12; A61L 2300/406; A61L 2300/216; A61L 2300/232; A61L 2300/25; A61L 2430/02

(57) ABSTRACT

Disclosed is a medicine loaded nano-capsule additive Polymethylmethacrylate (PMMA) cement and its production method.

### FIELD OF THE INVENTION

The invention relates to medicine loaded nano-capsule additive Polymethyl methacrylate (PMMA) cement and its production method.

The invention particularly relates to medicine loaded nano-capsule additive PMMA cement and its production method for use in orthopaedic surgery, especially in all fields wherein bone cement made from antibiotic additive PMMA used, such as osteomyelitis and periprostatic joint infection.

### BACKGROUND OF THE INVENTION

After inflammatory area is cleaned by bone infection surgery called osteomyelitis, antibiotic additive cement put in the area is brought into small beads and put in order like a surgical suture chain and left there for taking by another surgery operation to bone medulla after a few weeks. Through the same principle, in periprosthetic joint infection surgery, prosthesis is removed from the applied zone and after the zone is flushed macroscopically, space eliminators called spacer made from cement obtained from antibiotic loaded PMMA imitating shape of prosthesis is prepared and applied to the zone for cleaning the zone from micro-organisms at microscopic level before applying a new prosthesis. The spacer is kept in the zone until blood table and outside view of wound is fully recovered. Thus, local fighting against infection is also provided. Local fighting in said processes plays key role in period of systemic antibiotherapy and development of resistance against antibiotics. Applications used in the related field also bring some problems. The problems mentioned are summarised in items below:
Since there is no controlled release of the antibiotic, it is insufficient to fight infection as a result of its release at high levels in the first few days and at doses far below the therapeutic level in the following days. (Boelch vd. 2019, van Vugt vd. 2019)
All antibiotics not convenience for mixture with cement in antibiograms obtained from culture, only capable to add certain active agents into cement as antibiotics (van Vugt vd. 2019),
Capable to add antibiotics to cement at certain rates only (adding high dose of antibiotics may result in systemic toxic effects or adding antibiotics over a certain rate decreases resistance of cement (Boelch vd. 2017, Nodzo vd. 2017, Boelch vd. 2019),
The period specified by spaced in two stage revision surgeries varies between 3-10 months, upon decreasing of antibiotic release under therapeutic level after first few days, spacer acts as a foreign object that may cause development of infection (Nodzo vd. 2017, Wouthuyzen Bakker vd. 2019) (in the in-vitro studies, when measurable the lowest antibiotic concentration period and waiting period for infection in two staged revision operations are considered, an average of 3-9 months difference is seen.)
In case of inadequacy of local treatment in treatment process supported by systemic antibiotics, increase in side effects that can be seen in patients and treatment costs increase due to use of different antibiotics for longer times for systemic antibiotherapy (Boelch vd. 2019, van Vugt vd. 2019),
In case of being unsuccessful in fighting against infection, co-morbidities and patient's costs increase as a result of additional surgery operations (Gandhi vd. 2018, Siddiqi vd. 2019).

In conclusion, several problems and negativities as described above are experienced in the related method and present applications are insufficient for settlement of the problems and negativities. This case makes it necessary to make a development and novelty in the related method.

### BRIEF EXPLANATION OF THE INVENTION

The present invention relates to a medicine loaded nanocapsule additive PMMA cement meeting the needs mentioned above, eliminating all disadvantages and providing some additional advantages, and a production method for it.

Primary purpose of the invention is to provide protection of patients against infections by use of same therapeutic dose of Vancomycin (VCM) for longer period in comparison to methods available in the related field by means of standardizing effective dose by controlled release system.

Another purpose of the invention is to provide increase of cement resistance in comparison to cements in applications of the related field. The invention uses an activated capsuling polymer and thus provides high resistance.

Another purpose of the invention is to minimize antibiotic resistance risk by providing more effective fighting against infection.

A further purpose of the invention is to reduce quantity of antibiotics to be used and increase antibiotics efficiency when compared to present cement applications by use of synthesized nanocapsules.

Another purpose of the invention is to provide controlled VCM release from thermal sensitive nanocapsules due to increase of ambience temperature as a result of infection development.

A further purpose of the invention is to provide increase of VCM release from bacteria sensitive smart nanocapsules and accordingly achievement of effective antibiotic concentration for fighting against infection in case of occurrence of Staphylococcus aureus bacteria in ambience.

The structural and characteristics features and all advantages of the invention will be understood better with detailed descriptions given below. Therefore, the assessment should also be made taking into account the detailed description.

### DETAILED DESCRIPTION OF THE INVENTION

In this detailed description, the preferred applications of the invention have been described in a manner not forming any restrictive effect and only for purpose of better understanding of the matter.

The invention relates to medicine loaded nanocapsule additive PMMA cement and production method. The main agent for production of nanocapsules in the invention is a polymer with an encapsulation feature. The encapsulating polymers mentioned are triblock copolymers of conductive features and are fully biocompatible. Capsuling polymers used herein are activated by functional groups such as amine and carboxyl for use.

Nano capsule production under the invention is categorized in two as thermal sensitive and bacteria sensitive. In addition to encapsulating feature polymers, Hyaluronic acid (HA) is used to ensure effectivity in bacteria sensitive nanocapsule production. The HA in question is substrate of hyalurinidase enzyme released by Staphylococcus aureus bacteria. Increase of medicine release from antibiotic loaded nanocapsules is provided by help of HA. HA which is a substrate of Hyaluronidase enzyme secreted by Staphylococcus aureus bacteria is a type of polymer of bonding feature and is from glycosaminoglycans comprising anionic, long polysaccharides. They are known with general name of heteropolysaccharides which is a linear polymer comprising repetitive disaccharide units. For production of thermal sensitive nanocapsule, in addition to encapsulating feature polymers, cationic homopolymers capable to form loaded films easily by physical adsorption and electrostatic adsorption on various substrates including glass, metals, polymers and metallic oxides are used. Said cationic homopolymers which is basic material in thermal sensitive nanocapsule production provide opening and closing of polymer nanocapsule subject to temperature and adjustment of diffusion rate of medicine.

PMMA is the mixture with name of cement in orthopaedic surgery for several years as space filling or fixing agent in bone structure. In medicine loaded nanocapsule additive PMMA cement of the invention, PMMA is mixed with VCM. By use of method of the invention, tobramycin, gentamicin, linezolid and teicoplanin can also be used instead of Vancomycin for same purpose. Thus, local antibiotherapy effect is provided in the area of use.

In the medicine loaded nanocapsule additive PMMA cement production method of the invention, polymers of active encapsulating feature are prepared. In the next process step, thermal sensitive nanocapsules are synthesised by use of polymers/cationic homopolymers of encapsulating features. Then bacteria sensitive encapsulating feature polymers/hyaluronic acid base smart nanocapsules are synthesized. Synthesized thermal sensitive and bacteria sensitive nanocapsules have thermal sensitivity at range of 4° C. to 40° C. In addition, in order to provide dose control, in case of existence of Staphylococcus aureus in ambience, it comprises polymers/hyaluronic acid nanocapsules of smart capsuling feature comprising hyaluronidase enzyme substrate in about 90% of pathogen staphylococcus. Antibiotic release increases as a result of nano medicine release system interaction arising from hyaluronidase enzyme released in ambience having bacteria.

The bigger the size of thermal sensitive nanocapsule, the higher is wall permeability and the lower is the temperature. When temperature increases, nanocapsule size and permeability decreases. Since in stage of medicine loaded nanocapsule additive PMMA cement preparation, temperature reaches 60° C. to 80° C. for a short time period, nanocapsule permeability is decreased and VCM release is minimized. In this aspect, thermal sensitive nanocapsule use provides an advantage. After obtaining nanocapsules, VCM capsuling, incubation of nanocapsules in water solvent at low temperature and then removal of water and lyophilising and loading into the nanocapsules mentioned in a dialysis tube are performed. Average size of VCM loaded thermal sensitive nanocapsules is in range of 70-230 nm. This range is estimated taking into account the size in intra-cell applications.

Due to electrostatic interaction between HA and VCM in polymer/HA nanocapsules of encapsulating feature, it is targeted to achieve a higher encapsulating capability and release speed control in comparison to polymers/cationic homopolymer nanocapsules. The obtained medicine loaded nano capsules are contributed homogenously to standard PMMA polymer. In the mixing process mentioned here, preferably 1 g VCM loaded nanocapsule and 40 mg PMMA are used. Powder mixture obtained in the last process is mixed with monomer at 100 cycles/minute. As capsuling polymer Pluronic base polymers such as Pluronic®F127, Pluronic P123 or Pluronic F68 are used.

In order to solve above mentioned technical problems and achieve all advantages described in details below, the invention is a medicine loaded nanocapsule additive PMMA cement production method and comprises process steps of
a) Preparation of polymers of active capsuling feature,
b) Synthesizing thermal sensitive nanocapsules,
c) Synthesizing bacteria sensitive nanocapsules,
d) Loading VCM and/or tobramycin and/or gentamicin and/or linezolid and/or teicoplanin into said nanocapsule,
e) Loading loaded nanocapsules obtained in process step (d) into PMMA polymer.

1. A medicine loaded nanocapsule additive PMMA cement production method,
a) preparation of polymers of active capsuling feature,
b) synthesizing thermal sensitive nanocapsules,
c) synthesizing bacteria sensitive nanocapsules,
d) loading Vancomycin and/or tobramycin and/or gentamicin and/or linezolid and/or teicoplanin into nanocapsule in question, and
e) loading loaded nanocapsules obtained in process step (d) into Polymethylmethacrylate (PMMA) polymer.

2. The production method of claim 1, comprising synthesizing thermal sensitive nanocapsules from Pluronic® F127 and poly(ε-lysine) (ε-PL).

3. The production method of claim 1, and comprising synthesizing bacteria sensitive nanocapsules from Pluronic® F127 and Hyaluronic acid.

4. The production method of claim 1, comprising adding 1 gr Vancomycin loaded nanocapsule and 40 mg Polymethylmethacrylate (PMMA) polymer.
